(19) United States
(12) Patent Application Publication
(10) Pub. No.: US 2026/0048144 A1
(43) Pub. Date: Feb. 19, 2026

(54) SECRETED RNA THERAPEUTICS FOR NUCLEAR DELIVERY

(71) Applicant: H. Lee Moffitt Cancer Center and Research Institute, Inc., Tampa, FL (US)

(72) Inventors: Eric PADRON, Tampa, FL (US); Ariel QUINTANA-GONZALEZ, Wesley Chapel, FL (US)

(21) Appl. No.: 19/100,928

(22) PCT Filed: Aug. 3, 2023
(86) PCT No.: PCT/US2023/071583

§ 371 Date: Feb. 3, 2025

Related U.S. Application Data
(60) Provisional application No. 63/370,305, filed on Aug. 3, 2022.

(51) Int. Cl. A61K 48/00; A61K 38/16; A61K 38/45

(52) U.S. Cl. CPC A61K 48/005; A61K 38/162; A61K 38/45; C12Y 207/01021

(57) ABSTRACT

Disclosed herein is a system that addresses the current challenges described by combining three existing technologies and an improved nuclear RNA delivery system to generate a first-in-class cellular therapy that can secrete a cancer specific suicide gene for the treatment of several cancers. The system generally involves a first poly nucleotide encoding retroviral elements that preferentially binds and facilitates formation and secretion of vesicles carrying its own RNA messenger operably linked to a first expression control sequence; a second polynucleotide encoding an RNA therapeutic having a nuclear retention sequence that is flanked by packaging signals for the retroviral elements operably linked to a second expression control sequence; and a third polynucleotide encoding a membrane fusion protein operably linked to a third expression control sequence.

### CROSS-REFERENCE TO RELATED APPLICATIONS

This application claims benefit of U.S. Provisional Application No. 63/370,305, filed Aug. 3, 2022, which is hereby incorporated herein by reference in its entirety.

### SEQUENCE LISTING

This application contains a sequence listing filed in ST.26 format entitled “320805_2030_Sequence_Listing” created on Jul. 26, 2023, having 18,497 bytes. The content of the sequence listing is incorporated herein in its entirety.

### BACKGROUND

Suicide gene therapy refers to a therapeutic strategy whereby a modified genetic construct is introduced into cancer or immune cells designed to induce cell death (Bonini, C. et al. Mol Ther 2007 15:1248-1252; Duarte, S., et al. Cancer Lett 2012 324:160-170). When inserted directly in cancer cells, this strategy has conventionally aimed to leverage a local and, in some cases, distant ‘bystander effect’ (Rainov, N. G. Human gene therapy 2000 11:2389-2401; Freeman, S. M., et al. The Lancet 1997 349:2-3). This is because gene-delivery systems generally introduce the suicide construct in only a small fraction of the tumor bulk and therefore death of non-modified cancer cells is required for clinically meaningful efficacy. This reliance on a clinically unpredictable degree of ‘bystander effect’ has led to modest results in human trials (Rainov, N. G. Human gene therapy 2000 11:2389-2401). Further, suicide genes have generally been non-specific making in vivo clinically translatable strategies limited.

When inserted in immune cells, this strategy has been leveraged to minimize toxicity associated with genetically modified T-cells to include CAR-T therapy (Di Stasi, A. et al. New England Journal of Medicine 2011 365:1673-1683). Several delivery systems have been developed to introduce suicide gene therapy to include non-replicating and replicating viral vectors and the CRISPR/Cas9 system (Duarte, S., et al. Cancer Lett 2012 324:160-170; Di Stasi, A. et al. New England Journal of Medicine 2011 365:1673-1683). However, all of these strategies are limited by the relatively low efficiency of suicide gene delivery. Therefore, there exists a critical need for the development of novel therapeutic strategies that either improve the bystander effect or leverage other biologic mechanisms to maximize this approach.

### SUMMARY

Disclosed herein is a system that addresses all of the current challenges described above by combining three existing technologies and an improved nuclear RNA delivery system to generate a first-in-class cellular therapy that can secrete a cancer specific suicide gene for the treatment of several cancers.

Disclosed herein is a secreted RNA therapeutic system that involves
(a) a first polynucleotide encoding retroviral elements that preferentially binds and facilitates formation and secretion of vesicles carrying its own RNA messenger operably linked to a first expression control sequence;
(b) a second polynucleotide encoding an RNA therapeutic having a nuclear retention sequence that is flanked by packaging signals for the retroviral elements operably linked to a second expression control sequence; and
(c) a third polynucleotide encoding a membrane fusion protein operably linked to a third expression control sequence.

In some embodiments, the first, second and third polynucleotides are present in separate vectors. In some embodiments, the first, second and third polynucleotides are present in a single vector. In some embodiments, the delivery vesicle is a virus-like particle.

In some embodiments, the retroviral elements for forming a delivery vesicle comprises two or more of a retroviral gag protein, a retroviral envelope protein, a retroviral reverse transcriptase or a combination thereof. For example, the retroviral gag protein can be a gag-homology protein, such as paternally expressed gene 10 (PEG10). Therefore, in some embodiments, the packaging signals for the retroviral elements is the 5′ UTR and 3′ UTR for PEG10.

In some embodiments, the nuclear retention sequence is a portion of a long non-coding RNA (lncRNA) enriched in Alu repeats and/or a SIRLOIN motif

In some embodiments, the RNA therapeutic has a synthetic intron that can be spliced by a mutant spliceosome. For example, the RNA therapeutic can encode a herpes simplex virus thymidine kinase (HSV-TK) when the synthetic intron is spliced by a mutant spliceosome.

In some embodiments, the membrane fusion protein is the G envelope protein of vesicular stomatitis virus (VSV-G).

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates a system integrating a synthetic intron and PEG10 packaging.

FIG. 2 illustrates an embodiment of the disclosed system with a nuclear retention sequence (SEQ ID NO:8) as disclosed herein.

FIGS. 3A to 3C illustrates an embodiment of a Genomic Safe Harbor approach. FIG. 3A shows a trojan horse construct directed to the AVVS1 loci utilizing a Cas9 RNP with an sgRNA targeting AAVS1. FIG. 3B shows a second sgRNA arising from the same construct as the first sgRNA that targets a tumor suppressor for depletion leading to increased leukemic fitness. FIG. 3C shows an option where the sgRNA targeting the tumor suppressor is transcribed only when the amplicon is integrated ensuring suicide gene expression in those cells with increased fitness.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

### Systems for Delivering Cargo

Compositions, systems, and methods for delivering cargo to a target cell are described for example in WO2021055855A1, which is incorporated by reference in its entirety for these compositions, systems, and methods. These cargo delivering moieties are referred to herein as a “fusogen.”

In some embodiments, the compositions, systems, and methods for delivering cargo comprise one or more polynucleotides encoding one or more endogenous retroviral elements for forming a delivery vesicle and one or more capture moieties for packaging a cargo within the delivery vesicle. The one or more endogenous retroviral elements for forming a delivery vesicle may comprise two or more of a retroviral gag protein, a retroviral envelope protein, a retroviral reverse transcriptase or a combination thereof. The retroviral gag protein alone, the retroviral envelope protein alone, or both the retroviral gag protein and retroviral envelope protein may be endogenous.

In some embodiments, the envelope protein comprises a cargo-binding domain. In some embodiments, the cargo-binding domain is a hairpin loop-binding element. In some embodiments, the hairpin loop-binding element is an MS2 aptamer.

In some embodiments, the cargo comprises nucleic acids, proteins, a complex thereof, or a combination thereof. In some embodiments, the cargo is linked to one or more envelope proteins by a linker. In some embodiments, the linker is a glycine-serine linker. In some embodiments, the glycine-serine linker is (GGS) 3 (SEQ ID NO:7).

In some embodiments, the system further comprised a reverse transcriptase. In some embodiments, the one or more capture moieties comprise DNA-binding moieties, RNA-binding moieties, protein-binding moieties, or a combination thereof.

In some embodiments, the delivery vesicle is a virus-like particle (VLP). As used herein, the term “virus-like particle” (VLP) refers to a structure that in at least one attribute resembles a virus, but which has not been demonstrated to be infectious. A VLP may be a nonreplicating, noninfectious viral shell that contains a viral capsid but lacks all or part of the viral genome, in particular, the replicative components of the viral genome. VLPs are generally composed of one or more viral proteins, such as, but not limited to those proteins referred to as capsid, coat, shell, surface, and structural proteins (e.g., VPI, VP2). A VLP may also resemble the structure of a bacteriophage, being non-replicative and noninfectious, and lacking at least the gene or genes coding for the replication machinery of the bacteriophage, and also lacking the gene or genes encoding the protein or proteins responsible for viral attachment to or entry into the host.

In some embodiments, the system further comprises a targeting moiety, wherein the targeting moiety is capable of specifically binding to a target cell. In some embodiments, the targeting moiety comprises a membrane fusion protein. In some embodiments, the membrane fusion protein is the G envelope protein of vesicular stomatitis virus (VSV-G).

In some embodiments, the target cell is a mammalian cell. In some embodiments, the mammalian cell is a cancer cell. In some embodiments, the mammalian cell is infected with a pathogen. In some embodiments, the pathogen is a virus.

### Retroviral Gag Protein

Group-specific antigen (gag) proteins are the core structural proteins or the major components of the retroviral capsid. The HIV pl7 matrix protein (MA) is a 17 kDa protein, of 132 amino acids, which comprises the N-terminus of the Gag polyprotein. It is responsible for targeting Gag polyprotein to the plasma membrane but also makes contacts with the HIV trans membrane glycoprotein gp41 in the assembled virus and may play a critical role in recruiting Env glycoproteins to viral budding sites.

Several studies have shown that expression of the gag gene alone in a number of systems results in the efficient assembly and release of membrane enveloped virions (Craven, R. C., et al. (1996). Dynamic interactions of the Gag polyprotein. Current Topics in Microbiology and Immunology 214, pp. 65-94; Delchambre, M., et al. (1989). The Gag precursors of simian immunodeficiency virus assemble into virus-like particles. EMBO 8, pp. 2653-60; Dickson, C., et al. (1984). “Protein biosynthesis and assembly,” RNA tumor viruses (R. Weiss, N. Teich, H. Varmus, and J. Coffin, Eds.), Vol. 1, pp. 513-648. 2 vols. Cold Spring Harbor Laboratory, Cold Spring Harbor, N. Y.; Gheysen, H. P., et al. (1989), “Assembly and release of HIV-1 precursor Pr55gag virus-like particles from recombinant baculovirus-infected insect cells,” Cell 59, pp. 103-12; Haffar, O., et al. (1990), “Human immunodeficiency virus-like, non-replication, Gag-Env particles assemble in a recombinant vaccinia virus expression system,” J. Virol. 64, pp. 2653-59; Hunter, E. (1994), “Macromolecular interactions in the assembly of HIV and other retroviruses,” Sem. in Virology 5, pp. 71-83; Krausslich, H-G., et al. (1996), “Intracellular transport of retroviral capsid components,” Current Topics in Microbiology and Immunology 214, pp. 25-64; Madisen, L., et al. (1987), “Expression of the human immunodeficiency virus gag gene in insect cells,” Virology 158, pp. 248-250; Smith, A. J., et al. (1990), “Human immunodeficiency virus type 1 Pr55gag and Prl60gag-pol expressed from a simian virus 40 late-replacement vector are efficiently processed and assembled into virus-like particles,” J. Virol. 64, pp. 2743-50; Sommerfelt, M. A., et al. (1992), “Importance of the pl2 protein in Mason-Pfizer monkey virus assembly and infectivity,” J. Virol. 66, pp. 7005-11; Wills, J. W., et al. (1989), “Creation and expression of myristylated forms of Rous sarcoma virus Gag protein in mammalian cells,” J. Virol. 63, pp. 4331-43). Thus, the product of this gene has the necessary structural information to mediate intracellular transport, to direct assembly into the capsid shell, and to catalyze the process of membrane extrusion known as budding.

Once Gag is translated, Gag polyproteins are myristoylated at their N-terminal glycine residues by N-myristoyltransferase 1, a modification that is critical for plasma membrane targeting. In the membrane-unbound form, the MA myristoyl fatty acid tail is sequestered in a hydrophobic pocket in the core of the MA protein. Recognition of plasma membrane proteins by MA activates a “myristoyl switch”, wherein the myristoyl group is extruded from its hydrophobic pocket in MA and embedded in the plasma membrane.

The HIV nucleocapsid protein (NC) is a 7 kDa zinc finger protein in the Gag polyprotein and which, after viral maturation, forms the viral nucleocapsid. NC recruits full-length viral genomic RNA to nascent virions.

The neuronal gene Arc bears homology to the Gag component of Ty3/gypsy retrotransposons and exhibits biochemical properties that are reminiscent of retroviral Gag proteins. The Arc protein assembles into virus-like capsids both in cells and when recombinantly expressed in bacteria. Arc capsids are able to encapsulate their own mRNA, mediating their intercellular transfer in extracellular vesicles. Purified Arc proteins may be used to reconstitute capsids with different DNA or RNA or proteins or some mixture thereof and can be packaged into the capsid for delivery into cells. In some embodiments, capsids may be assembled using lipids to aid uptake by cells. Various embodiments may utilize different Arc orthologs.

In some embodiments, the polynucleotides described herein may comprise a Gag-homology protein or functional domain thereof. The term “functional domain” refers to a polypeptide sequence that has an activity other than binding to the nucleic acid sequence recognized by the nucleic acid-binding domain. By combining a nucleic acid-binding domain with one or more effector domains, the polypeptides of the invention may be used to target the one or more functions or activities mediated by the effector domain to a particular target DNA sequence to which the nucleic acid-binding domain specifically binds.

Molecular and genetic determinants of Gag-mediated intercellular communication may be determined by characterizing the mechanisms of capsid-mediated intercellular mRNA transfer, with particular focus on features that could enable use of this system for programmable delivery of cargo. Different Gag proteins evolved diverse RNA-binding domains for mediating specific encapsidation of their RNA genomes. The RNA-binding sequence specificity of the human Gag homology proteins can be tested through protein pull down and sequencing of associated RNA and/or through sequencing of the extracellular vesicle fraction from HEK293 cells that over-express each protein. The nucleic-acid-binding domains can be swapped between proteins, or additional RNA-binding domains with known specificity can be fused to test the extent to which binding specificity can be reprogrammed. Accordingly, the Gag-homology protein or functional domain thereof can comprise both the export compartment domain and nucleic acid-binding complain.

The Gag-homology protein can be selected from Arc, ASPRV1, a Sushi-Class protein, a SCAN protein, or a PNMA protein. In particular instances, the Gag-homology protein is a PNMA protein, for example, ZCC18, ZCH12, PNM8B, PNM6A, PNMA6E_i2, PMA6F, PMAGE, PNMAI, PNMA2, PNM8A, PNMA3, PNMA4, PNMA5, PNMA6, PNMA7, PNMAI, MOAPI, or CCD8. In embodiments, the Gag-homology protein is an Arc protein, in certain embodiments, hARC or dARCI. The Gag-homology protein can comprise ASPRV1. In other instances, the Gag-homology protein is PEG10, RTL3, RTL10, or RTL1. In certain embodiments, the Gag Homology protein is a SCAN protein, for example, PGBD1. In instances, the PEG10 Gag homology protein is PEG10_i6 or PEG10_i2.

In some embodiments, the Gag-homology protein or functional domain thereof may comprise both the export compartment domain and the nucleic acid-binding domain. In specific embodiments, the nucleic acid binding-domain may be modified relative to the native nucleic acid-binding domain of the Gag-homology protein. In specific embodiments, the nucleic acid binding domain may be a non-native nucleic acid-binding domain relative to the Gag-homology protein. In some embodiments, the Gag-homology protein may be Arc or a paraneoplastic Ma antigen (PNMA) protein.

In some embodiments, the recombinant GAG-like proteins may be expressed and purified from bacteria, yeast, insect cells, or mammalian cells. The recombinant GAG-like proteins may be purified under denaturing conditions and transferred to non-denaturing conditions by buffer exchange.

In some embodiments, the retroviral gag protein is endogenous. In some embodiments, the retroviral gag protein may contain the NC and MA domains. In some embodiments, the retroviral gag protein may be a gag-homology protein, as described herein.

In some embodiments, the gag-homology protein may include, but is not necessarily limited to, Arcl, Asprvl, PNMA1, PNMA3, PNMA4, PNMA5, PNMA6, PNMA7, PEG10, RTL1, MOAPI, or ZCCHC12. In specific embodiments, the gag-homology protein is Arcl, PNMA6a, or PNMA3. In specific embodiments, the gag-homology protein is PEG10. In some embodiments, the gag-homology protein may contain a DNA-binding motif. As a specific example, and as discussed in Example 4, PEG10 comprises a DNA binding motif that allows for packaging of DNA of specific sequences.

As any person of skill in the art would appreciate, any of the systems described herein can be further engineered to a minimal set of components and be applied to any suitable endogenous element. As described in Examples 3 and 4 and FIGS. 56-70, use of PEG10 is just an example approach that can be followed with any other endogenous element.

### Retroviral Env Protein

Env is a retroviral gene that encodes the protein that forms the viral envelope. The expression of the env gene allows retroviruses to target and attach to specific cell types, and to infiltrate the target cell membrane. The structure and sequence of several different env genes suggests that Env proteins are type 1 fusion machines. Type 1 fusion machines initially bind a receptor on the target cell surface, which triggers a conformational change, allowing for binding of the fusion protein. The fusion peptide inserts itself in the host cell membrane and brings the host cell membrane very close to the viral membrane, allowing for membrane fusion. The sequence of the env gene may differ significantly between retroviruses, however, the gene is always located downstream of gag, pro, and pol. The env mRNA has to be spliced to be expressed.

Env not only mediates virus entry into cells, but is also a major target for both cellular and antibody responses. It is synthesized as a precursor molecule, gpl60, which is subsequently processed into the surface subunit (SU) gpl20 and the transmembrane subunit (TM) gp41 by a cellular protease, and exists as a trimer of gpl20-gp41 heterodimers on viral or cell membranes. The SU protein domain determines the tropism of the virus because it is responsible for the receptor-binding function of the virus. The SU domain therefore determines the specificity of the virus for a single receptor molecule. gpl20 interacts with receptor and coreceptor molecules for HIV and mediates virus attachment to the cell, while gp41 causes subsequent fusion between viral and cell membranes for releasing viral core components into the cell during the initial infection process. The TM protein consists of three distinct domains: the extracellular domain, the transmembrane domain, and the cytoplasmic domain.

In some embodiments, the retroviral envelope protein is endogenous. In some embodiments, the envelope protein may be from a Gammaretrovirus. In some embodiments, the envelope protein may be from a Deltaretrovirus. In some embodiments, the envelope protein may be selected from, but is not necessarily limited to, envHI, envH2, envH3, envKI, envK2_I, envK2_2, envK3, envK4, envK5, envK6, envT, envW, envWI, envfrd, envR (b), envR, envF(c)2, or envF(c)I.

In some embodiments, the envelope protein may comprise a cargo-binding domain. In some embodiments, the cargo-binding domain is a hairpin loop-binding element. In some embodiments, the hairpin loop-binding element is an MS2 aptamer.

In some embodiments, the retroviral gag protein and the retroviral envelope protein are both endogenous. In some embodiments, the gag protein is endogenous and the envelope protein is of viral origin. In some embodiments, the envelope protein is endogenous and the gag protein is of viral origin.

### Capture Moieties

In some embodiments, the vesicles comprise one or more capture moieties, e.g., for packaging a cargo and/or recruiting specific cargo(s) into the vesicle. The term “nucleic acid capture moiety” or simply “capture moiety”, as used herein, refers to a moiety which binds selectively to a target molecule. Optionally, the moiety can be immobilized on an insoluble support, as in a microarray or to microparticles, such as beads. When used as a primer, a probe of the invention would likely not be anchored to a solid support. A capture moiety can “capture” a target molecule by hybridizing to the target and thereby immobilizing the target. In cases wherein the moiety itself is immobilized, the target too becomes immobilized. Such binding to a solid support may be through a linking moiety, which is bound to either the capture moiety or to the solid support.

The capture moiety may comprise one or more genes endogenous to the polynucleotide or plasmid, for example genes capable of recruiting the plasmid into the vesicle. The capture moiety may comprise exogenous genes or may comprise molecules capable of recruiting or capturing cargo molecules for the vesicles. In some examples, the capture moieties may interact with the cargo. The capture moieties may be nucleic acid-binding molecules, e.g., DNA, RNA, DNA-binding proteins, RNA-binding proteins, or a combination thereof. In some embodiments, the capture moieties may be protein-binding molecules, e.g., DNA, RNA, antibodies, nanobodies, antigens, receptors, ligands, fragments thereof, or a combination thereof. The capture moieties can be fused to endogenous genes or exogenous genes.

In some embodiments, the one or more capture moieties comprise DNA-binding moieties, RNA-binding moieties, protein-binding moieties, or a combination thereof.

In certain embodiments, the capture moiety may be labelled, as with, e.g., a fluorescent moiety, a radioisotope (e.g., 32P), an antibody, an antigen, a lectin, an enzyme (e.g., alkaline phosphatase or horseradish peroxidase, which can be used in calorimetric methods), chemiluminescence, bioluminescence or other labels well known in the art. In certain embodiments, binding of a target strand to a capture moiety can be detected by chromatographic or electrophoretic methods. In embodiments in which the capture moiety does not contain a detectable label, the target nucleic acid sequence may be so labelled, or, alternatively, labelled secondary probes may be employed. A “secondary probe” includes a nucleic acid sequence which is complementary to either a region of the target nucleic acid sequence or to a region of the capture moiety. Region G of a probe (which will most often not be complementary to the target), might be useful in capturing a secondary labelled nucleic acid probe.

In some embodiments, the capture moiety is a nucleic acid hairpin. The terms “nucleic acid hairpin”, “hairpin capture moiety”, or simply “hairpin”, as used herein, refer to a unimolecular nucleic acid-containing structure which comprises at least two mutually complementary nucleic acid regions such that at least one intramolecular duplex can form. Hairpins are described in, for example, Cantor and Schimmel, “Biophysical Chemistry”, Part III, p. 1183 (1980). In certain embodiments, the mutually complementary nucleic acid regions are connected through a nucleic acid strand; in these embodiments, the hairpin comprises a single strand of nucleic acid. A region of the capture moiety which connects regions of mutual complementarity is referred to herein as a “loop” or “linker”. In some embodiments, a loop comprises a strand of nucleic acid or modified nucleic acid. In some embodiments, the linker is not a hydrogen bond. In other embodiments, the loop comprises a linker region which is not nucleic-acid-based; however, capture moieties in which the loop region is not a nucleic acid sequence are referred to herein as hairpins. Examples of non-nucleic-acid linkers suitable for use in the loop region are known in the art and include, for example, alkyl chains (see, e.g., Doktycz et al. (1993) Biopolymers 33:1765). While it will be understood that a loop can be a single-stranded region of a hairpin, for the purposes of the discussion below, a “single-stranded region” of a hairpin refers to a non-loop region of a hairpin. In embodiments in which the loop is a nucleic acid strand, the loop preferably comprises 2-20 nucleotides, more preferably 3-8 nucleotides. The size or configuration of the loop or linker is selected to allow the regions of mutual complementarity to form an intramolecular duplex. In preferred embodiments, hairpins useful in the present invention will form at least one intramolecular duplex having at least 2 base-pairs, more preferably at least 4 base-pairs, and still more preferably at least 8 base-pairs. The number of base-pairs in the duplex region, and the base composition thereof can be chosen to assure any desired relative stability of duplex formation. For example, to prevent hybridization of non-target nucleic acids with the intramolecular duplex-forming regions of the hairpin, the number of base-pairs in the intramolecular duplex region will generally be greater than about 4 base-pairs. The intramolecular duplex will generally not have more than about 40 base-pairs. In preferred embodiments, the intramolecular duplex is less than 30 base-pairs, more preferably less than 20 base-pairs in length.

A hairpin may be capable of forming more than one loop. For example, a hairpin capable of forming two intramolecular duplexes and two loops is referred to herein as a “double hairpin”. In preferred embodiments, a hairpin will have at least one single-stranded region which is substantially complementary to a target nucleic acid sequence. “Substantially complementary” means capable of hybridizing to a target nucleic acid sequence under the conditions employed. In preferred embodiments, a “substantially complementary” single-stranded region is exactly complementary to a target nucleic acid sequence. In preferred embodiments, hairpins useful in the present invention have a target-complementary single-stranded region having at least 5 bases, more preferably at least 8 bases. In preferred embodiments, the hairpin has a target-complementary single-stranded region having fewer than 30 bases, more preferably fewer than 25 bases. The target-complementary region will be selected to ensure that target strands form stable duplexes with the capture moiety. In embodiments in which the capture moiety is used to detect target strands from a large number of non-target sequences (e.g., when screening genomic DNA), the target-complementary region should be sufficiently long to prevent binding of non-target sequences. A target-specific single-stranded region may be at either the 3′ or the 5′ end of the capture moiety strand, or it may be situated between two intramolecular duplex regions (for example, between two duplexes in a double hairpin).

Also disclosed is a delivery vesicle comprising one or more components encoded in the one or more polynucleotides in the engineered delivery system described herein. In some embodiments, the one or more components of the delivery vesicle comprise two or more of a retroviral gag protein, a retroviral envelope protein, a retroviral reverse transcriptase, or a combination thereof.

In some embodiments, the Gag-homology protein or functional domain thereof may comprise both the export compartment domain and the nucleic acid-binding domain. In specific embodiments, the nucleic acid binding-domain may be modified relative to the native nucleic acid-binding domain of the Gag-homology protein. In specific embodiments, the nucleic acid binding domain may be a non-native nucleic acid-binding domain relative to the Gag-homology protein. In some embodiments, the Gag-homology protein may be Arc or a paraneoplastic Ma antigen (PNMA) protein.

In some embodiments, the vesicle comprises a cell-specific targeting moiety. In some embodiments, the cell-specific targeting moiety targets a mammalian cell. In some embodiments, the cell-specific targeting moiety comprises a membrane fusion protein. In some embodiments, the membrane fusion protein is VSV-G.

In some embodiments, the mammalian cell is a cancer cell. In some embodiments, the mammalian cell is infected with a pathogen. In some embodiments, the pathogen is a virus.

### Synthetic Introns

In some embodiments, the RNA therapeutic is a synthetic intron. For example, artificial nucleic acid introns configured for selective splicing in cells with aberrant RNA splicing activity, e.g., neoplastic cells, are disclosed in WO2022087427A1, which is incorporated by reference in its entirety for these introns and methods of use thereof.

In some embodiments, the artificial intron can comprise a 5′ splice site, a canonical 3′ splice site, at least one cryptic 3′ splice site, a pyrimidine-rich domain, and at least one branchpoint. Also provided are constructs integrating the artificial introns with exons in a configuration that, when the artificial intron is spliced out by the aberrant RNA splicing factors, encode a functional protein.

In some embodiments, the disclosure provides an artificial nucleic acid construct comprising an intron. The intron comprises: a 5′ splice site; a canonical 3′ splice site; at least one cryptic 3′ splice site, that is within about 100 nucleotides upstream of the canonical 3′ splice site or within about 50 nucleotides downstream of the canonical 3′ splice site; a pyrimidine-rich domain comprising at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; and at least one branchpoint at least 15 nucleotides upstream of the canonical 3′ splice site.

In some embodiments, the intron is at least about 50 nucleotides to about 1000 nucleotides in length.

In some embodiments, the intron is derived from a human wildtype intron selected from intron 1 of MTERFD3, intron 4 of MY015B, intron 10 of SYTL1, intron 11 of SYTL1, intron 4 of MAP3K7, intron 1 of ORAI2, and intron 1 of TMEM14C. In some embodiments, the intron is derived from a human wildtype intron 1 of MTERFD3, and wherein the intron further comprises one, two, three, or more of the following features: a 5′ splice site comprising a GT dinucleotide immediately followed by a consensus 5′ splice site context, optionally wherein the consensus 5′ splice site context includes one of AAG, GAG, GTG, and the like; a canonical 3′ splice site comprising an AG dinucleotide immediately preceded by a C or T; at least one cryptic 3′ splice site, located at least 5 nucleotides upstream of the canonical 3′ splice site, with an AG dinucleotide and comprising a sequence that is a weaker 3 splice site than is the canonical 3 splice site, where splice site strength is estimated with the MaxEntScan algorithm or similar methods; a pyrimidine-rich domain comprising at least 15 consecutive nucleotides, wherein the sequence of the pyrimidine-rich domain is at least 60% pyrimidine nucleotides and at least 40% thymine nucleotides, and wherein the pyrimidine-rich domain is within at least 30 nucleotides of a cryptic 3′ splice site; and at least one branchpoint at least 20 nucleotides upstream of the canonical 3′ splice site. In some embodiments, the intron has a 5′ end domain with about 10 to about 150 nucleotides having at least 50% sequence identity to a sequence of the 5′-most 10 to about 150 nucleotides of the wildtype intron. In some embodiments, the intron has a 3′ end domain with about 50 to about 350 nucleotides having at least 50% sequence identity to a sequence of the 3′-most 50 to about 350 nucleotides of the wildtype intron.

In some embodiments, the 5′ splice site comprises a sequence selected from GTGAG, GTAAG, GTGCG, GTACG, GTGGG, GTAGG, GTGTG, GTATG, and GTATC. In some embodiments, the canonical 3′ splice site comprises a sequence selected from AAG, CAG, and TAG. In some embodiments, the at least one cryptic 3′ splice site comprises a sequence selected from AAG, CAG, GAG, TAG, ATG, CTG, GTG, and TTG. In some embodiments, the intron comprises a plurality of cryptic 3′ splice sites within about 100 nucleotides upstream of the canonical 3′ splice site or within about 100 nucleotides downstream of the canonical 3′ splice site, and wherein each of the plurality of the cryptic 3′ splice sites comprises a sequence independently selected from AAG, CAG, GAG, TAG, ATG, CTG, GTG, and TTG.

In some embodiments, the at least one branchpoint is at least 20 nucleotides upstream of the canonical 3′ splice site, and wherein the branchpoint nucleotide is an adenine. In some embodiments, the branchpoint and surrounding sequence context has sequence identity of at least 60% to the sequence tactaAca, where the uppercase A is the branchpoint nucleotide.

In some embodiments, the intron is configured to be spliced differently in a cancer cell comprising a change-of-function or loss-of-function mutation in a recurrently mutated RNA splicing factor gene relative to the splicing pattern of the intron in a cell lacking a change-of-function or loss-of-function mutation in a recurrently mutated RNA splicing factor gene. In some embodiments, the RNA splicing factor gene is SF3B1. In some embodiments, the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M7841, E802Q, M971T, M971V, and combinations thereof, with reference to the wild-type amino acid sequence.

In some embodiments, the nucleic acid construct further comprises a first exon domain and a second exon domain, wherein the intron is disposed between the first exon domain and the second exon domain. In some embodiments, the combination of the first exon domain and the second exon domain without the intron encodes part or all of a protein of interest. In some embodiments, the nucleic acid intron construct comprises an expression cassette comprising the first exon domain, the intron, the second exon domain, and a promoter sequence operatively linked thereto.

In some embodiments, the disclosure provides a method of generating an artificial nucleic acid construct with an intron, e.g., an artificial intron. The method comprises:
(1) ligating a 5′ end domain of a human wildtype intron to a 3′ end domain of the human wildtype intron to provide an abbreviated intron that lacks an interior sequence, wherein the 5′ end domain comprises about 10 to about 150 nucleotides of the 5′ end sequence of the human wildtype intron, and wherein the 3′ end domain comprises about 50 to about 350 nucleotides of the 3′ end sequence of the human wildtype intron;
(2) implementing one or more sequence modifications to the abbreviated intron sequence to provide a first plurality of artificial introns derived from the abbreviated intron sequence;
(3) selecting artificial introns from the first plurality of artificial introns that conform to at least three of the following parameters: a 5 splice site; a canonical 3′ splice site; at least one cryptic 3′ splice site, that is within about 100 nt nucleotides upstream of the canonical 3′ splice site or within about 50 nt nucleotides downstream of the canonical 3′ splice site; a pyrimidine-rich domain comprising at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; and at least one branchpoint at least 15 nucleotides upstream of the canonical 3′ splice site.

In some embodiments, the one or more sequence modifications comprises one or more of the following in any combination or order: (a) mutating a single nucleotide; (b) mutating any pair of nucleotides within 10 nucleotides of the 5′ end of the abbreviated intron sequence or 30 nucleotides of the 3′ end of the abbreviated intron sequence; (c) deleting any consecutive stretch of 5, 10, 15, 20, 25, 30, 35, 40, 45, 50, 100, 125, 150, 200, or 250 nucleotides; (d) mutating any pair of nucleotides within the 5 nt nucleotides upstream of and 2 nucleotides downstream of each branchpoint; (e) mutating any combination of branchpoints to guanine; (f) mutating any combination of multiple adenines to guanines; (g) mutating any combination of branchpoint contexts to strong branchpoint contexts, optionally wherein the strong branchpoint context comprises a sequence with a sequence identity of at least 50% to the sequence tactaAca, where A is a branchpoint nucleotide and tacta_ca is a context sequence; (h) mutating any four consecutive nucleotides to cAGg; (i) inserting a polypynmidine tract immediately followed by a 3 splice site at any position; (j) mutating any consecutive stretch of nucleotides to one or more thymines; (k) mutating all pyrimidines within any six or more consecutive positions to guanines; (1) inserting a strong branchpoint and flanking sequence context at any position; (m) inserting one or more intronic splicing enhancers at any position; and (n) inserting one or more intronic splicing silencers at any position.

In some embodiments, the polypyrimidine tract immediately followed by a 3′ splice site comprises at least 6 consecutive nucleotides containing at least 4 pyrimidines, immediately followed by a sequence selected from AAG, CAG, GAG, TAG, ATG, CTG, GTG, or TTG, and the like. In some embodiments, the strong branchpoint and flanking sequence context comprises a sequence with a sequence identity of at least 50% to the sequence tactaAca, where uppercase indicates the branchpoint, and the like. In some embodiments, the one or more intronic splicing enhancers are selected from GGGTTT, GGTGGT, TTTGGG, GAGGGG, GGTATT, GTAACG, and the like. In some embodiments, the one or more intronic splicing silencers are selected from C AC ACC A, CTCCTC, TACAGCT, CTTCAG, GAACAG, CAAAGGA, AGATATT, ACATGA, AATTTA, AGTAGG, and the like.

In some embodiments, the method further comprises introducing the modified target nucleic acid molecule to a cancer cell with a mutation in an RNA splicing factor gene and permitting expression, or alternately selective lack of expression, of the protein of interest.

In some embodiments, the target nucleic acid molecule is a gene in the chromosome of a cell, wherein the gene encodes a protein of interest, and the modified target nucleic acid molecule is configured for selective expression, or alternately selective lack of expression, in a cell characterized by a mutation in an RNA splicing factor gene. In some embodiments, the cell is a cancer cell and the mutation in an RNA splicing factor gene is a change-of-function or loss-of-function mutation in a recurrently mutated RNA splicing factor gene; wherein the artificial intron sequence is configured to be spliced differently in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene; wherein the different splicing pattern of the artificial intron sequence results in production of different mature transcripts of the modified target nucleic acid molecule in a cancer cell comprising the change-of-function or loss-of-function mutation in the recurrently mutated RNA splicing factor gene, relative to the splicing pattern of the intron in a cell lacking the change-of-function or loss-of-function mutation in the recurrently mutated RNA splicing factor gene; and wherein the production of different mature transcripts of the modified nucleic acid molecule permits either selective expression, or alternately selective lack of expression, of a desired protein from the target nucleic acid molecule in the cancer cell, and the opposite pattern in a cell lacking the change-of-function or loss-of-function mutation in the recurrently mutated RNA splicing factor gene. In some embodiments, the RNA splicing factor gene is SF3B1.

In some embodiments, upon splicing of the at least one artificial nucleic acid intron from the gene transcript in a cancer cell the CDS encodes a functional therapeutic protein. In some embodiments, the functional therapeutic protein is a suicide gene, toxin, chemokine, cytokine, growth factor, targetable cell-surface protein, targetable antigen, druggable enzyme, detectable marker, and the like.

In some embodiments, the functional therapeutic protein is a chemokine, cytokine, or growth factor, and wherein the chemokine, cytokine, or growth factor stimulates an increased immune response against the cancer cell.

In some embodiments, the functional therapeutic protein is a druggable enzyme. A druggable enzyme is an enzyme that is ideally not substantially prevalent in healthy cells, but when expressed presents a target for a known therapeutic, which can be additionally administered to the specific detriment of the cancer cell expressing the druggable enzyme target. Various druggable enzymes and their associated therapeutics are known and are encompassed by this disclosure. Non-limiting examples are provided below.

In one embodiment, the druggable enzyme is herpes simplex virus thymidine kinase and the method further comprises administering to the subject an effective amount of ganciclovir. For example, a CDS for herpes simplex virus thymidine kinase (HSV-TK) can be divided by the disclosed artificial introns in an expression cassette. When transcribed and properly spliced in leukemic and melanoma cells with a change of function mutation in the RNA splicing factor gene SF3B1, the exons are combined in the mRNA leading to proper expression of the HSV-TK protein in the cells. Upon treatment with ganciclovir, the cells are selectively killed compared to cells not properly expressing the HSV-TK (i.e., cell not receiving the expression cassette or cells with receiving the expression cassette but having wild-type SF3B1).

In one embodiment, the druggable enzyme is cytosine deaminase and the method further comprises administering to the subject an effective amount of 5-fluorocytosine. In one embodiment, the druggable enzyme is nitroreductase and the method further comprises administering to the subject an effective amount of CB1954 or analogs thereof. In one embodiment, the druggable enzyme is carboxypeptidase G2 and the method further comprises administering to the subject an effective amount of CMDA, ZD-2767P, and the like. In one embodiment, the druggable enzyme is purine nucleoside phosphorylase and the method further comprises administering to the subject an effective amount of 6-methylpurine deoxyriboside, and the like. In one embodiment, the druggable enzyme is cytochrome P450 and the method further comprises administering to the subject an effective amount of cyclophosphamide, ifosfamide, and the like. In one embodiment, the druggable enzyme is horseradish peroxidase and the method further comprises administering to the subject an effective amount of indole-3-acetic acid, and the like. In one embodiment, the druggable enzyme is carboxylesterase and the method further comprises administering to the subject an effective amount of irinotecan, and the like.

### RNA Therapeutics

In some embodiments, the RNA therapeutic is a mRNA, sgRNA, siRNA, shRNA, miRNA, tRNA, rRNA, snRNA, or lncRNA. The polynucleotides may include natural nucleotides, modified nucleotides, analogs of natural nucleotides, such as labeled nucleotides, or any combination thereof.

In some embodiments, the RNA therapeutic is a genetic modulating agent. In some embodiments, the genetic modulating agent comprises one or more components of a gene editing system and/or polynucleotides encoding thereof. In some embodiments, the gene editing system is a CRISPR-Cas system. In some embodiments, the CRISPR-Cas system is a Type II, Type V, or Type VI CRISPR-Cas system. In some embodiments, the Type II CRISPR-Cas system comprises CRISPR-Cas9. In some embodiments, the Type V CRISPR-Cas system comprises CRISPR-Casl2. In some embodiments, the Type VI CRISPR-Cas system comprises CRISPR-Casl3. In some embodiments, a Cas protein of the CRISPR-Cas system may be modified to bind to a binding domain of the envelope protein. In some embodiments, a guide molecule of the CRISPR-Cas system is modified to bind to a binding domain of the envelope protein. In some embodiments, the modification comprises incorporation of a hairpin loop that binds to a hairpin-binding element on the envelope protein. In some embodiments, the hairpin loop may be recognized by the MS2 aptamer.

### Nuclear Targeting Sequences

In some embodiments, the disclosed RNA therapeutics further comprise a nuclear retention RNA sequence, such as a nuclear retention sequence derived from naturally occurring RNAs that are exclusively found in the nucleus. Examples of nuclear retention sequences are described for example in Lubelsky, Y. & Ulitsky, I. Nature 2018 555:107-111 and Trenkmann, M, et al. Nature Reviews Genetics 2018 19:188-189, which are incorporated by reference in its entirety for the teaching of these sequences.

In some embodiments, the nuclear retention sequence is a long non-coding RNA (lncRNA) enriched in Alu repeats and/or SIRLOIN motif. For example, in some embodiments, the nuclear retention sequence is metastasis-associated lung adenocarcinoma transcript 1 (MALAT1). In some embodiments, the nuclear retention sequence is

In some embodiments, the retention sequence is present on the 3′ end of the RNA therapeutic.

### Specific Embodiments

In some embodiments, the packaging signals for the retroviral elements comprises 5′ UTR and 3′ UTR for PEG10. In some embodiments, the 5′UTR comprises the nucleic acid sequence tcctcggtgcaacctatataaggctcacagtctgcgctcctggtacacgcgcttcaacttcggttggtgtgtgtcgaagaaacc tgactgcgccctgaggagaacagcggagaaggtccaccgagcctggcgaaaggtccgctgagcgggctgtcgtccgg agccactccgggctgcggagcacccagtggagaccgcgcctggctcaggtgtgggaccccatccttcctgtcttcgcaga ggagtcctcgcgtgaaataagcgggttttgaaaacaaaaaaaagaaggagtggaagagggggccaggatccaggcct ccatccccacagaagtgaagctacagctgggaggtctcctcccaccccaaccgtcaccctgggtcccgactgcccacctc ctcctcctccccctccccccaacaacaacaacaacaacaactccaagcacaccggccataagagtgcgtgtgtccccaa c (SEQ ID NO:1). In some embodiments, the 3′UTR comprises the nucleic acid sequence

(SEQ ID NO: 2)
atacctgtcatgtccttcaggatctctgccctcaaaatttattcctgttc
agcttctcaatcagtgactgtgtgctaaattttaggctactgtatcttca
ggccacctgaggcacatcctctctgaaacggctatggaaggttagggcca
ctctggactggcacacatcctaaagcaccaaaagaccttcaacattttct
gagagcaacagagtatttgccaataaatgatctctcatttttccaccttg
actgccaatctaactaaaataattaataagtttactttccagccagtcct
ggaagtctgggttttacctgccaaaacctccatcaccatctaaattatag
gctgccaaatttgctgtttaacatttacagagaagctgatacaaacgcag
gaaatgctgatttctttatggagggggagacgaggaggaggaggacatga
cttttcttgcggtttcggtaccctctttttaaatcactggaggactgagg
ccttattaaggaagc.

In some embodiments, the RNA therapeutic encodes a herpes simplex virus thymidine kinase (HSV-TK) when the synthetic intron is spliced by a mutant spliceosome. In some embodiments, the spliced HSV-TK gene has the nucleic acid sequence

(SEQ ID NO: 3)
gcttcgtacccctgccatcaacacgcgtctgcgttcgaccaggctgcgcg
ttctcgcggccatagcaaccgacgtacggcgttgcgccctcgccggcagc
aagaagccacggaagtccgcctggagcagaaaatgcccacgctactgcgg
gtttatatagacggtcctcacgggatggggaaaaccaccaccacgcaact
gctggtggccctgggttcgcgcgacgatatcgtctacgtacccgagccga
tgacttactggcaggtgctgggggcttccgagacaatcgcgaacatctac
accacacaacaccgcctcgaccagggtgagatatcggccggggacgcggc
ggtggtaatgacaagcgcccagataacaatgggcatgccttatgccgtga
ccgacgccgttctggctcctcatatcgggggggaggctgggagctcacat
gccccgcccccggccctcaccctcatcttcgaccgccatcccatcgccgc
cctcctgtgctacccggccgcgcgataccttatgggcagcatgacccccc
aggccgtgctggcgttcgtggccctcatcccgccgaccttgcccggcaca
aacatcgtgttgggggcccttccggaggacagacacatcgaccgcctggc
caaacgccagcgccccggcgagcggcttgacctggctatgctggccgcga
ttcgccgcgtttacgggctgcttgccaatacggtgcggtatctgcagggc
ggcgggtcgtggcgggaggattggggacagctttcggggacggccgtgcc
gccccagggtgccgagccccagagcaacgcgggcccacgaccccatatcg
gggacacgttatttaccctgtttcgggcccccgagttgctggcccccaac
ggcgacctgtacaacgtgtttgcctgggccttggacg.

In some embodiments, the HSV-TK gene containing the synthetic intron has the nucleic acid sequence

(SEQ ID NO: 4)
gcttcgtacccctgccatcaacacgcgtctgcgttcgaccaggctgcgcg
ttctcgcggccatagcaaccgacgtacggcgttgcgccctcgccggcagc
aagaagccacggaagtccgcctggagcagaaaatgcccacgctactgcgg
gtttatatagacggtcctcacgggatggggaaaaccaccaccacgcaact
gctggtggccctgggttcgcgcgacgatatcgtctacgtacccgagccga
tgacttactggcaggtgctgggggcttccgagacaatcgcgaacatctac
accacacaacaccgcctcgaccagggtgagatatcggccggGTGAGTCGC
CCCCTTCCTCTGCTCCGAGAGGGAAATGGGAATTAGGGTGGTGGCAGAGC
CCAAAGAGGCCTTGTAGGCCATGGTAAGGCATTTCTATGTTTTATTTTAC
TTTGTCTTTATCCTAAAATGCCATTGGCAAGTTTATTGCAGggacgcggc
ggtggtaatgacaagcgcccagataacaatgggcatgccttatgccgtga
ccgacgccgttctggctcctcatatcgggggggaggctgggagctcacat
gccccgcccccggccctcaccctcatcttcgaccgccatcccatcgccgc
cctcctgtgctacccggccgcgcgataccttatgggcagcatgacccccc
aggccgtgctggcgttcgtggccctcatcccgccgaccttgcccggcaca
aacatcgtgttgggggcccttccggaggacagacacatcgaccgcctggc
caaacgccagcgccccggcgagcggcttgacctggctatgctggccgcga
ttcgccgcgtttacgggctgcttgccaatacggtgcggtatctgcagggc
ggcgggtcgtggcgggaggattggggacagctttcggggacggccgtgcc
gccccagggtgccgagccccagagcaacgcgggcccacgaccccatatcg
gggacacgttatttaccctgtttcgggcccccgagttgctggcccccaac
ggcgacctgtacaacgtgtttgcctgggccttggacg

In some embodiments, the nuclear retention sequence has the nucleic acid sequence tacccatacgatottccagattacgct (SEQ ID NO:5).

Therefore, in some embodiments, the secreted RNA therapeutic system has the nucleic acid sequence

(SEQ ID NO: 6)
gacggatcgggagatctcccgatcccctatggtgcactctcagtacaatctgctctgatgccgcatagt
taagccagtatctgctccctgcttgtgtgttggaggtcgctgagtagtgcgcgagcaaaatttaagcta
caacaaggcaaggcttgaccgacaattgcatgaagaatctgcttagggttaggcgttttgcgctgcttc
gcgatgtacgggccagatatacgcgttgacattgattattgactagttattaatagtaatcaattacgg
ggtcattagttcatagcccatatatggagttccgcgttacataacttacggtaaatggcccgcctggct
gaccgcccaacgacccccgcccattgacgtcaataatgacgtatgttcccatagtaacgccaataggga
ctttccattgacgtcaatgggtggagtatttacggtaaactgcccacttggcagtacatcaagtgtatc
atatgccaagtacgccccctattgacgtcaatgacggtaaatggcccgcctggcattatgcccagtaca
tgaccttatgggactttcctacttggcagtacatctacgtattagtcatcgctattaccatggtgatgc
ggttttggcagtacatcaatgggcgtggatagcggtttgactcacggggatttccaagtctccacccca
ttgacgtcaatgggagtttgttttggcaccaaaatcaacgggactttccaaaatgtcgtaacaactccg
ccccattgacgcaaatgggcggtaggcgtgtacggtgggaggtctatataagcagagctctctggctaa
ctagagaacccactgcttactggcttatcgaaattaatacgactcactatagggagataatacgactca
ctatagcccaagctggctagcgtttaaaacctcctcggtgcaacctatataaggctcacagtctgcgct
cctggtacacgcgcttcaacttcggttggtgtgtgtcgaagaaacctgactgcgccctgaggagaacag
cggagaaggtccaccgagcctggcgaaaggtccgctgagcgggctgtcgtccggagccactccgggctg
cggagcacccagtggagaccgcgcctggctcaggtgtgggaccccatccttcctgtcttcgcagaggag
tcctcgcgtgaaataagcgggttttgaaaacaaaaaaaagaaggagtggaagagggggccaggatccag
gcctccatccccacagaagtgaagctacagctgggaggtctcctcccaccccaaccgtcaccctgggtc
ccgactgcccacctcctcctcctccccctccccccaacaacaacaacaacaacaactccaagcacaccg
gccataagagtgcgtgtgtccccaacgccaccatgtacccatacgatgttccagattacgcttcgtacc
catacgatgttccagattacgctgaagcgtccGCTTCGTACCCCTGCCATCAACACGCGTCTGCGTTCG
ACCAGGCTGCGCGTTCTCGCGGCCATAGCAACCGACGTACGGCGTTGCGCCCTCGCCGGCAGCAAGAAG
CCACGGAAGTCCGCCTGGAGCAGAAAATGCCCACGCTACTGCGGGTTTATATAGACGGTCCTCACGGGA
TGGGGAAAACCACCACCACGCAACTGCTGGTGGCCCTGGGTTCGCGCGACGATATCGTCTACGTACCCG
AGCCGATGACTTACTGGCAGGTGCTGGGGGCTTCCGAGACAATCGCGAACATCTACACCACACAACACC
GCCTCGACCAGGGTGAGATATCGGCCGGGTGAGTCGCCCCCTTCCTCTGCTCCGAGAGGGAAATGGGAA
TTAGGGTGGTGGCAGAGCCCAAAGAGGCCTTGTAGGCCATGGTAAGGCATTTCTATGTTTTATTTTACT
TTGTCTTTATCCTAAAATGCCATTGGCAAGTTTATTGCAGGGACGCGGCGGTGGTAATGACAAGCGCCC
AGATAACAATGGGCATGCCTTATGCCGTGACCGACGCCGTTCTGGCTCCTCATATCGGGGGGGAGGCTG
GGAGCTCACATGCCCCGCCCCCGGCCCTCACCCTCATCTTCGACCGCCATCCCATCGCCGCCCTCCTGT
GCTACCCGGCCGCGCGATACCTTATGGGCAGCATGACCCCCCAGGCCGTGCTGGCGTTCGTGGCCCTCA
TCCCGCCGACCTTGCCCGGCACAAACATCGTGTTGGGGGCCCTTCCGGAGGACAGAC
ACATCGACCGCCTGGCCAAACGCCAGCGCCCCGGCGAGCGGCTTGACCTGGCTA
TGCTGGCCGCGATTCGCCGCGTTTACGGGCTGCTTGCCAATACGGTGCGGTATCT
GCAGGGCGGCGGGTCGTGGCGGGAGGATTGGGGACAGCTTTCGGGGACGGCCG
TGCCGCCCCAGGGTGCCGAGCCCCAGAGCAACGCGGGCCCACGACCCCATATC
GGGGACACGTTATTTACCCTGTTTCGGGCCCCCGAGTTGCTGGCCCCCAACGGC
GACCTGTACAACGTGTTTGCCTGGGCCTTGGACGagccccaagaagaagagaaaggtgga
ggccagctaaatacctgtcatgtccttcaggatctctgccctcaaaatttattcctgttcagcttctca
atcagtgactgtgtgctaaattttaggctactgtatcttcaggccacctgaggcacatcctctctgaaa
cggctatggaaggttagggccactctggactggcacacatcctaaagcaccaaaagaccttcaacattt
tctgagagcaacagagtatttgccaataaatgatctctcatttttccaccttgactgccaatctaacta
aaataattaataagtttactttccagccagtcctggaagtctgggttttacctgccaaaacctccatca
ccatctaaattataggctgccaaatttgctgtttaacatttacagagaagctgatacaaacgcaggaaa
tgctgatttctttatggagggggagacgaggaggaggaggacatgacttttcttgcggtttcggtaccc
tctttttaaatcactggaggactgaggccttattaaggaagccttaagcttggtaccgagctcggatcc
actagtccagtgtggtggaattctgcagatatccagcacagtggcggccgctcgagtctagagggcccg
tttaaacccgctgatcagcctcgactgtgccttctagttgccagccatctgttgtttgcccctcccccg
tgccttccttgaccctggaaggtgccactcccactgtcctttcctaataaaatgaggaaattgcatcgc
attgtctgagtaggtgtcattctattctggggggtggggtggggcaggacagcaagggggaggattggg
aagacaatagcaggcatgctggggatgcggtgggctctatggcttctgaggcggaaagaaccagctggg
gctctagggggtatccccacgcgccctgtagcggcgcattaagcgcggcgggtgtggtggttacgcgca
gcgtgaccgctacacttgccagcgccctagcgcccgctcctttcgctttcttcccttcctttctcgcca
cgttcgccggctttccccgtcaagctctaaatcgggggctccctttagggttccgatttagtgctttac
ggcacctcgaccccaaaaaacttgattagggtgatggttcacgtagtgggccatcgccctgatagacgg
tttttcgccctttgacgttggagtccacgttctttaatagtggactcttgttccaaactggaacaacac
tcaaccctatctcggtctattcttttgatttataagggattttgccgatttcggcctattggttaaaaa
atgagctgatttaacaaaaatttaacgcgaattaattctgtggaatgtgtgtcagttagggtgtggaaa
gtccccaggctccccagcaggcagaagtatgcaaagcatgcatctcaattagtcagcaaccaggtgtgg
aaagtccccaggctccccagcaggcagaagtatgcaaagcatgcatctcaattagtcagcaaccatagt
cccgcccctaactccgcccatcccgcccctaactccgcccagttccgcccattctccgccccatggctg
actaattttttttatttatgcagaggccgaggccgcctctgcctctgagctattccagaagtagtgagg
aggcttttttggaggcctaggcttttgcaaaaagctcccgggagcttgtatatccattttcggatctga
tcaagagacaggatgaggatcgtttcgcatgattgaacaagatggattgcacgcaggttctccggccgc
ttgggtggagaggctattcggctatgactgggcacaacagacaatcggctgctctgatgccgccgtgtt
ccggctgtcagcgcaggggcgcccggttctttttgtcaagaccgacctgtccggtgccctgaatgaact
gcaggacgaggcagcgcggctatcgtggctggccacgacgggcgttccttgcgcagctgtgctcgacgt
tgtcactgaagcgggaagggactggctgctattgggcgaagtgccggggcaggatctcctgtcatctca
ccttgctcctgccgagaaagtatccatcatggctgatgcaatgcggcggctgcatacgcttgatccggc
tacctgcccattcgaccaccaagcgaaacatcgcatcgagcgagcacgtactcggatggaagccggtct
tgtcgatcaggatgatctggacgaagagcatcaggggctcgcgccagccgaactgttcgccaggctcaa
ggcgcgcatgcccgacggcgaggatctcgtcgtgacccatggcgatgcctgcttgccgaatatcatggt
ggaaaatggccgcttttctggattcatcgactgtggccggctgggtgtggcggaccgctatcaggacat
agcgttggctacccgtgatattgctgaagagcttggcggcgaatgggctgaccgcttcctcgtgcttta
cggtatcgccgctcccgattcgcagcgcatcgccttctatcgccttcttgacgagttcttctgagcggg
actctggggttcgaaatgaccgaccaagcgacgcccaacctgccatcacgagatttcgattccaccgcc
gccttctatgaaaggttgggcttcggaatcgttttccgggacgccggctggatgatcctccagcgcggg
gatctcatgctggagttcttcgcccaccccaacttgtttattgcagcttataatggttacaaataaagc
aatagcatcacaaatttcacaaataaagcatttttttcactgcattctagttgtggtttgtccaaactc
atcaatgtatcttatcatgtctgtataccgtcgacctctagctagagcttggcgtaatcatggtcatag
ctgtttcctgtgtgaaattgttatccgctcacaattccacacaacatacgagccggaagcataaagtgt
aaagcctggggtgcctaatgagtgagctaactcacattaattgcgttgcgctcactgcccgctttccag
tcgggaaacctgtcgtgccagctgcattaatgaatcggccaacgcgcggggagaggcggtttgcgtatt
gggcgctcttccgcttcctcgctcactgactcgctgcgctcggtcgttcggctgcggcgagcggtatca
gctcactcaaaggcggtaatacggttatccacagaatcaggggataacgcaggaaagaacatgtgagca
aaaggccagcaaaaggccaggaaccgtaaaaaggccgcgttgctggcgtttttccataggctccgcccc
cctgacgagcatcacaaaaatcgacgctcaagtcagaggtggcgaaacccgacaggactataaagatac
caggcgtttccccctggaagctccctcgtgcgctctcctgttccgaccctgccgcttaccggatacctg
tccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatcc
ggtaactatcgtcttgagtccaacccggtaagacacgacttatcgccactggcagcagccactggtaac
aggattagcagagcgaggtatgtaggcggtgctacagagttcttgaagtggtggcctaactacggctac
actagaagaacagtatttggtatctgcgctctgctgaagccagttaccttcggaaaaagagttggtagc
tcttgatccggcaaacaaaccaccgctggtagcggtttttttgtttgcaagcagcagattacgcgcaga
aaaaaaggatctcaagaagatcctttgatcttttctacggggtctgacgctcagtggaacgaaaactca
cgttaagggattttggtcatgagattatcaaaaaggatottcacctagatccttttaaattaaaaatga
agttttaaatcaatctaaagtatatatgagtaaacttggtctgacagttaccaatgcttaatcagtgag
gcacctatctcagcgatctgtctatttcgttcatccatagttgcctgactccccgtcgtgtagataact
acgatacgggagggcttaccatctggccccagtgctgcaatgataccgcgagacccacgctcaccggct
ccagatttatcagcaataaaccagccagccggaagggccgagcgcagaagtggtcctgcaactttatcc
gcctccatccagtctattaattgttgccgggaagctagagtaagtagttcgccagttaatagtttgcgc
aacgttgttgccattgctacaggcatcgtggtgtcacgctcgtcgtttggtatggcttcattcagctcc
ggttcccaacgatcaaggcgagttacatgatcccccatgttgtgcaaaaaagcggttagctccttcggt
cctccgatcgttgtcagaagtaagttggccgcagtgttatcactcatggttatggcagcactgcataat
tctcttactgtcatgccatccgtaagatgcttttctgtgactggtgagtactcaaccaagtcattctga
gaatagtgtatgcggcgaccgagttgctcttgcccggcgtcaatacgggataataccgcgccacatagc
agaactttaaaagtgctcatcattggaaaacgttcttcggggcgaaaactctcaaggatcttaccgctg
ttgagatccagttcgatgtaacccactcgtgcacccaactgatcttcagcatcttttactttcaccagc
gtttctgggtgagcaaaaacaggaaggcaaaatgccgcaaaaaagggaataagggcgacacggaaatgt
tgaatactcatactcttcctttttcaatattattgaagcatttatcagggttattgtctcatgagcgga
tacatatttgaatgtatttagaaaaataaacaaataggggttccgcgcacatttccccgaaaagtgcca
cctgacgtc,
with PEG10 sequences in lowercase BOLD, the HSV-TK gene in uppercase
BOLD, the synthetic intron in uppercase, an SV40 NLS in lowercase
italics, and an HA sequence in lowercase italics and bold.

### Methods of Use

Also disclosed are methods of targeting an RNA therapeutic to the nucleus of a cell using the disclosed systems. In particular, the disclosed system packages the RNA therapeutic into a vesicle (e.g. VLP) for delivery to a cell. The disclosed nuclear retention signal targets the RNA therapeutic to the cell nucleus. In some embodiments, the RNA therapeutic is a suicide gene or druggable enzyme having a synthetic intron, such that the suicide gene or druggable enzyme is not functional unless the spliced by a mutant spliceosome, e.g. in a cancer cell.

Therefore, also disclosed is a method of treating cancer in a subject that involves administering to the subject a therapeutically effective amount of a system disclosed herein the RNA therapeutic is a suicide gene or druggable enzyme having a synthetic intron, such that the suicide gene or druggable enzyme is not functional unless the spliced by a mutant spliceosome in the cancer cells.

In some embodiments, the cancer is a myelodysplastic syndrome (MDS), chronic myelomonocytic leukemia (CMML), chronic lymphocytic leukemia (CLL), acute myeloid leukemia (AML), uveal melanoma, mucosal melanoma, skin melanoma, breast cancer, pancreatic cancer, endometrial cancer, liver cancer, lung cancer, mesothelioma, or other neoplasm with recurrent SF3B1 mutations. In some embodiments, upon splicing of the at least one artificial nucleic acid intron from the gene transcript, the gene of interest encodes a functional therapeutic protein. In some embodiments, the functional therapeutic protein is a toxin, chemokine, cytokine, growth factor, targetable cell-surface protein, targetable antigen, druggable enzyme, detectable marker, and the like.

In another aspect, the disclosure provides a method of treating in a subject with cancer, wherein the cancer is characterized by a change-of-function or loss-of-function mutation in a recurrently mutated RNA splicing factor gene. The method comprises administering to the subject an effective amount of a therapeutic composition comprising an expression cassette comprising a coding sequence (CDS) interrupted by at least one artificial nucleic acid intron as described herein, wherein the expression cassette further comprises a promoter operatively linked to the CDS.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Several suicide gene constructs have been generated each with distinct advantages. These include systems that convert prodrugs to active cytotoxic agents (i.e. Tsk/GSV system) (Fillat, C., et al. Current gene therapy 2003 3:13-26), systems that render cells sensitive to a monoclonal antibody with minimal toxicity (i.e. rituximab system) (Serafini, M. et al. Human gene therapy 2004 15:63-76), and systems that render cells sensitive to otherwise biologically inert compounds (i.e. iCasp9 system) (Di Stasi, A. et al. New England Journal of Medicine 2011 365:1673-1683). Disclosed herein is a system that addresses all of the current challenges described above by combining three existing technologies and an improved nuclear RNA delivery system to generate a first-in-class cellular therapy that can secrete a cancer specific suicide gene for the treatment of several cancers. Each technology is now integrated into a proof-of-concept cellular product.

### Engineering a Cellular Factory for the Secretion of a Suicide Gene:

Using the recently described PEG10 system, CAR-T cells were previously generated with the capacity to secrete RNA-containing virus like particles with the capacity to efficiently enter target cells (Segel, M. et al. Science 2021 373:882-889). This system has now been engineered to instead secrete the below, cancer specific suicide gene construct.

### a Splicing Mutation Specific Suicide Gene for the Treatment of Blood Cancers:

Mutations in the splicing machinery occur very frequently in myeloid and lymphoid cancer. The consequence of this is a slightly altered consensus sequencing for spliceosome binding and redefined intron/exon boundaries. To therapeutically exploit these ‘change of function’ mutations present only in cancer cells, a ‘synthetic intron’ was developed that is only spliced by mutant spliceosome components (North, K. et al. Nature biotechnology 2022 40:1103-1113). This synthetic intron has been inserted in a suicide gene such that only spliceosome mutant cells have the capacity to fully translate this to a functional protein and succumb to this therapy. Using a state-of-the-art secretion system, the disclosed proof-of-concept cellular product has now integrated a SF3B1 specific suicide gene that is secreted to its microenvironment.

a Nuclear Targeting Signal Derived from Naturally Occurring RNA Sequences:

The splicing machinery is only present in the nucleus and current RNA medicines and delivery systems, including the synthetic intron approach and PEG10 system utilized here, lack the capacity for nuclear translocation. While extremely innovative, this limitation prevents clear clinical translation of this technology for patients. To address this known nuclear retention RNA sequences derived from naturally occurring RNAs that are exclusively found in the nucleus, such as MALAT1, were investigated (Lubelsky, Y. & Ulitsky, I. Nature 2018 555:107-111). The optimal location for the nuclear targeting sequence on the RNA medicine was identified, demonstrating that a synthetic intron containing GFP construct could only be translated if the sequence was present on the 3′ position. The disclosed proof-of-concept cellular product contains this synthetic intron with the so-called SIRLOIN sequence at the 3′ position (Lubelsky, Y. & Ulitsky, I. Nature 2018 555:107-111). However, other retention signals are being optimized. Therefore, disclosed herein is the repurposing a nuclear retention sequence for nuclear targeting of RNA medicines.

a Second Safe Harbor Suicide Gene for the Generation of a by-Standard Effect in Cancers:

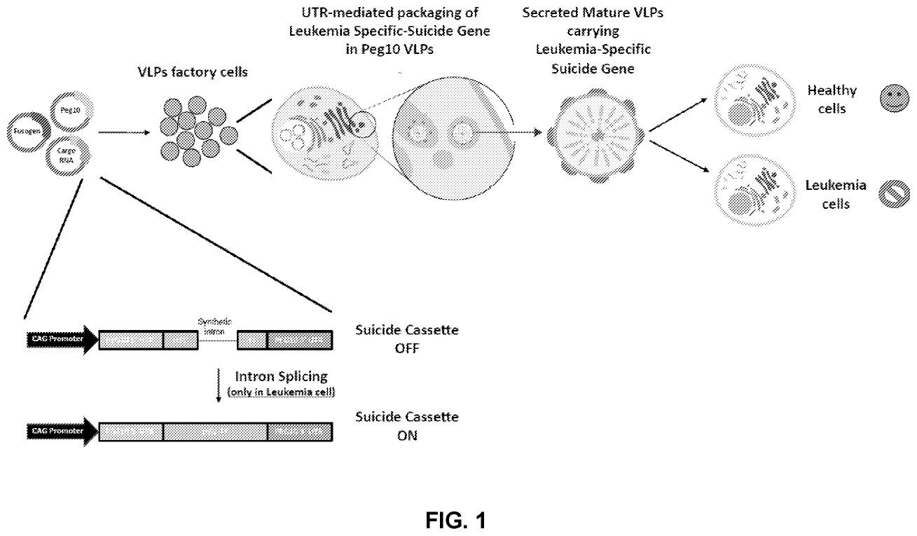

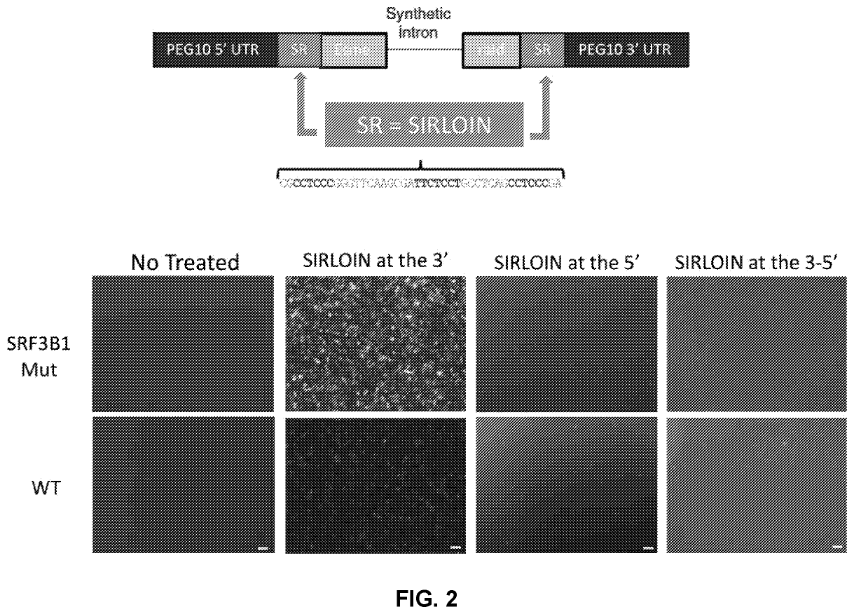

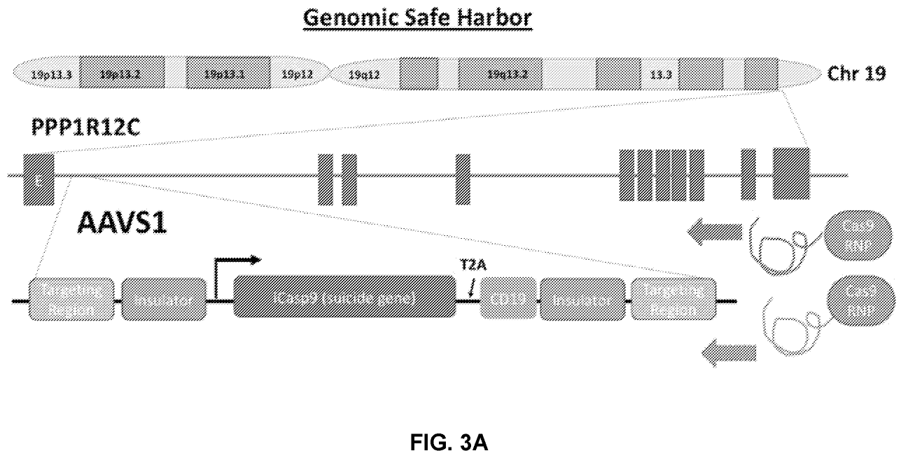

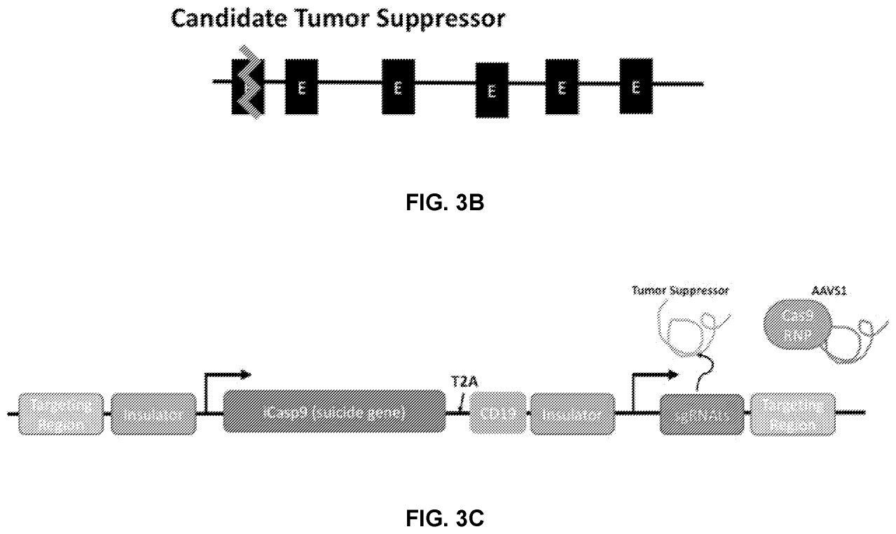

Last, a second suicide gene was inserted that is transcribed but only activated with the addition of systemic therapy. While this approach can be applied to CAR-T cells, the current proof-of-concept cellular product is a genetically engineered human leukemia myeloid cell line. This has two specific advantages: (1) there a redundant evolutionarily conserved homing mechanisms present that may result in better bone marrow to CAR-T cells; and (2) if myeloid penetration relative cellular product is induced towards programmed cell death, a therapeutically relevant by-standard effect may be achieved. To potentially leverage this a construct that renders cells universally sensitive to AP1903 was have inserted, an otherwise biologically inert compound, already tested and deemed safe without adverse effects in humans (Di Stasi, A. et al. New England Journal of Medicine 2011 365:1673-1683). A similar construct is also generated using rituximab (Serafini, M. et al. Human gene therapy 2004 15:63-76), instead of AP1903, as adaptive therapy (Budde, L. E. et al. PloS one 2013 8: e82742). Rituximab has been infused in thousands of lymphoma and rheumatoid arthritis patients with a favorable side effect profile (Coiffier, B. et al. New England Journal of Medicine 2002 346, 235-242; Lopez-Olivo, M. A., et al. Rituximab for rheumatoid arthritis. The Cochrane Library 2015). Of note, this construct has been inserted in the AAVS1 safe harbor site (Ordovas, L. et al. Stem cell reports 2015 5:918-931), to prevent undesirable off target genomic events (FIG. 1A) and has been flanked with insulator sequences to prevent epigenetic silencing as previously described (Fu, H. et al. Nature biotechnology 2006 24:572-576). Alternatively, the suicide gene could be inserted using CRISPR targeting approaches within a tumor suppressor gene to increase replicative fitness of our target in vivo (FIG. 1B-1C).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A secreted RNA therapeutic system, comprising
(a) a first polynucleotide encoding retroviral elements that preferentially binds and facilitates formation and secretion of vesicles carrying its own RNA messenger operably linked to a first expression control sequence;
(b) a second polynucleotide encoding an RNA therapeutic having a nuclear retention sequence that is flanked by packaging signals for the retroviral elements operably linked to a second expression control sequence; and
(c) a third polynucleotide encoding a membrane fusion protein operably linked to a third expression control sequence.

2. The system of claim 1, wherein the retroviral elements for forming a delivery vesicle comprises two or more of a retroviral gag protein, a retroviral envelope protein, a retroviral reverse transcriptase or a combination thereof.

3. The system of claim 2, wherein the retroviral gag protein is a gag-homology protein.

4. The system of claim 3, wherein the gag-homology protein is Paternally expressed gene 10 (PEG10).

5. The system of claim 1, wherein the nuclear retention sequence is a portion of a long non-coding RNA (lncRNA) enriched in Alu repeats and/or a SIRLOIN motif.

6. The system of claim 5, wherein the nuclear retention sequence has the nucleic acid sequence SEQ ID NO:5.

7. The system of claim 1, wherein the RNA therapeutic comprises a synthetic intron.

8. The system of claim 7, wherein the RNA therapeutic encodes a herpes simplex virus thymidine kinase (HSV-TK) when the synthetic intron is spliced by a mutant spliceosome.

9. The system of claim 8, wherein the RNA therapeutic comprises the nucleic acid sequence SEQ ID NO:4.

10. The system of claim 1, wherein the packaging signals for the retroviral elements comprises 5′ UTR and 3′ UTR for PEG10.

11. The system of claim 1, wherein the membrane fusion protein is the G envelope protein of vesicular stomatitis virus (VSV-G).

12. The system of claim 1, wherein the first, second and third polynucleotides are present in separate vectors.

13. The system of claim 1, wherein the first, second and third polynucleotides are present in a single vector.

14. The system of claim 13, having the nucleic acid sequence SEQ ID NO:6.

15. The system of claim 1, wherein the delivery vesicle is a virus-like particle.


SEQUENCE LISTING (8 sequences)

SEQ ID NO: 1 - DNA, 478 bp, synthetic construct
  source 1..478: mol_type = other DNA
tcctcggtgcaacctatataaggctcacagtctgcgctcctggtacacgcgcttcaacttcggttggtgtgtgtcgaagaaacctgactgcgccctgaggagaacagcggagaaggtccaccgagcctggcgaaaggtccgctgagcgggctgtcgtccggagccactccgggctgcggagcacccagtggagaccgcgcctggctcaggtgtgggaccccatccttcctgtcttcgcagaggagtcctcgcgtgaaataagcgggttttgaaaacaaaaaaaagaaggagtggaagagggggccaggatccaggcctccatccccacagaagtgaagctacagctgggaggtctcctcccaccccaaccgtcaccctgggtcccgactgcccacctcctcctcctccccctccccccaacaacaacaacaacaacaactccaagcacaccggccataagagtgcgtgtgtccccaac

SEQ ID NO: 2 - DNA, 515 bp, synthetic construct
  source 1..515: mol_type = other DNA
atacctgtcatgtccttcaggatctctgccctcaaaatttattcctgttcagcttctcaatcagtgactgtgtgctaaattttaggctactgtatcttcaggccacctgaggcacatcctctctgaaacggctatggaaggttagggccactctggactggcacacatcctaaagcaccaaaagaccttcaacattttctgagagcaacagagtatttgccaataaatgatctctcatttttccaccttgactgccaatctaactaaaataattaataagtttactttccagccagtcctggaagtctgggttttacctgccaaaacctccatcaccatctaaattataggctgccaaatttgctgtttaacatttacagagaagctgatacaaacgcaggaaatgctgatttctttatggagggggagacgaggaggaggaggacatgacttttcttgcggtttcggtaccctctttttaaatcactggaggactgaggccttattaaggaagc

SEQ ID NO: 3 - DNA, 937 bp, synthetic construct
  source 1..937: mol_type = other DNA
gcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacg

SEQ ID NO: 4 - DNA, 1087 bp, synthetic construct
  source 1..1087: mol_type = other DNA
gcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccgggtgagtcgcccccttcctctgctccgagagggaaatgggaattagggtggtggcagagcccaaagaggccttgtaggccatggtaaggcatttctatgttttattttactttgtctttatcctaaaatgccattggcaagtttattgcagggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacg

SEQ ID NO: 5 - DNA, 27 bp, synthetic construct
  source 1..27: mol_type = other DNA
tacccatacgatgttccagattacgct

SEQ ID NO: 6 - DNA, 7640 bp, synthetic construct
  source 1..7640: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagataatacgactcactatagcccaagctggctagcgtttaaaacctcctcggtgcaacctatataaggctcacagtctgcgctcctggtacacgcgcttcaacttcggttggtgtgtgtcgaagaaacctgactgcgccctgaggagaacagcggagaaggtccaccgagcctggcgaaaggtccgctgagcgggctgtcgtccggagccactccgggctgcggagcacccagtggagaccgcgcctggctcaggtgtgggaccccatccttcctgtcttcgcagaggagtcctcgcgtgaaataagcgggttttgaaaacaaaaaaaagaaggagtggaagagggggccaggatccaggcctccatccccacagaagtgaagctacagctgggaggtctcctcccaccccaaccgtcaccctgggtcccgactgcccacctcctcctcctccccctccccccaacaacaacaacaacaacaactccaagcacaccggccataagagtgcgtgtgtccccaacgccaccatgtacccatacgatgttccagattacgcttcgtacccatacgatgttccagattacgctgaagcgtccgcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccgggtgagtcgcccccttcctctgctccgagagggaaatgggaattagggtggtggcagagcccaaagaggccttgtaggccatggtaaggcatttctatgttttattttactttgtctttatcctaaaatgccattggcaagtttattgcagggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgagccccaagaagaagagaaaggtggaggccagctaaatacctgtcatgtccttcaggatctctgccctcaaaatttattcctgttcagcttctcaatcagtgactgtgtgctaaattttaggctactgtatcttcaggccacctgaggcacatcctctctgaaacggctatggaaggttagggccactctggactggcacacatcctaaagcaccaaaagaccttcaacattttctgagagcaacagagtatttgccaataaatgatctctcatttttccaccttgactgccaatctaactaaaataattaataagtttactttccagccagtcctggaagtctgggttttacctgccaaaacctccatcaccatctaaattataggctgccaaatttgctgtttaacatttacagagaagctgatacaaacgcaggaaatgctgatttctttatggagggggagacgaggaggaggaggacatgacttttcttgcggtttcggtaccctctttttaaatcactggaggactgaggccttattaaggaagccttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - protein, 9 aa, synthetic construct
GGSGGSGGS

SEQ ID NO: 8 - DNA, 42 bp, synthetic construct
  source 1..42: mol_type = other DNA
cgcctcccgggttcaagcgattctcctgcctcagcctcccga